(19) United States
(12) Patent Application Publication
(10) Pub. No.: US 2010/0236634 A1
(43) Pub. Date: Sep. 23, 2010

(54) Method of Formation of Hydrate Particles in a Water-Containing Hydrocarbon Fluid Flow

(75) Inventors: Sven Nuland, Strommen (NO); Martin Foss, Skedsmokorset (NO)

Correspondence Address: SUTHERLAND ASBILL & BRENNAN LLP, 999 PEACHTREE STREET, N.E., ATLANTA, GA, 30309, US

(73) Assignee: INSTITUTT FOR ENERGITEKNIKK, Kjeller (NO)

(21) Appl. No.: 12/739,410

(22) PCT Filed: Oct. 27, 2008
(86) PCT No.: PCT/NO08/00379

§ 371 Date: Apr. 23, 2010

(30) Foreign Application Priority Data
Oct. 25, 2007 (NO) 20075424

(51) Int. Cl. F17D 1/16; C07D 307/04; C01G 5/02; C01G 23/047; C01F 7/02

(52) U.S. Cl. 137/13; 549/429; 423/491; 423/610; 423/625

(57) ABSTRACT

The present invention relates to a method of adding nucleation seeds in order to promote the formation of hydrate particles in a flow containing hydrocarbon fluids and water. The method may include adding nucleation seeds at a point in the flow before the hydrate-forming area, the nucleation seeds being available for the promotion of the hydrate formation when this area is reached. The invention also relates to use of various compounds for the promotion of hydrate formation in such a fluid flow, thereby forming a transportable hydrate slurry.

Present application relates to a method for preventing blocking by formation of hydrates and more specific a method to prevent blocking of the production by formation of hydrates in hydrocarbon/water-mixtures in subsea pipelines.

Hydrate formation is a serious problem, especially by transport of hydrocarbons in pipelines from offshore oilfields to onshore facilities or to other floating facilities. Hydrate formation is a serious problem by production of oil and gas, since it can produce blockings which can stop the production and require costly solution to avoid and remove such blockings.

Hydrates consist of water which forms a solid phase in the presence of certain gas molecules (e.g. methane, carbon dioxide, etc) at high pressure and sufficiently low temperature, but above the normal conditions for ice formation.

The hydrate formation process is perceived by imagining water molecules in a cage-like structure. At temperatures below the freezing point of water, these structures are stable and no ice is formed. At temperature above the freezing point, the thermal movements of the molecules will overcome the forces between the water molecules and the structure will dissolve. However, if a gas molecule is present within the cage-structure, the additional forces between the gas molecule and the water molecules are sufficient to stabilise the structure and an ice-like substance is formed. Thus the limit for hydrate formation will be dependent on the forces between the gas molecule and the water molecules. Certain gases are therefore capable of forming gas hydrates at much higher temperatures and lower pressure than natural gas, e.g. sulphur hexafluoride or certain Freon gases.

There is usually a certain time between a gas/water mixture when the conditions for hydrate formation until the actual formation of the hydrates. A certain super cooling is necessary to initiate the hydrate formation. When the hydrates initially start to form, this will often occur on the pipe wall, since this is the coldest area of the subsea pipeline, and the hydrate that without measures to moderate the hydrate structure could be in the form of a solid, ice-like substance can block the pipeline. When pressure and temperature lie within the hydrate formation area, then the hydrate can form if appropriate condensation seed are present.

To solve the problem connected to hydrate formation, several prior solutions are known.

The traditional method to solve the hydrate problem has been to add chemicals that reduce the temperature for hydrate formation to below the system temperature. These chemicals are denoted as thermo dynamic inhibitors, and can be e.g. methanol, glycerol, etc. The inhibitors are required in large quantities, which imply logistic problems, in addition to considerable costs for the chemicals itself. By increasing the amount of non-hydrocarbon fluid to be transported, this will imply an additional, undesirable pressure drop, which can lead to a reduction of the capacity of the transport system, e.g. by reducing the maximum distance a multi-phase transport pipe can operate, without put into effect a pressure increase. This limitation gets more severe at increasing water depths. In addition it may be necessary with additional treatment onshore.

A more recent approach implies a low dosage of hydrate inhibitors which can be divided into two different kinds: kinetic inhibitors and anti agglomerants. The latter is the most relevant in relation to present invention.

An anti agglomerant allows formation of hydrates, but in the form of small particles. These can be transported with the flow, if the flow rate is sufficiently high, and blocking can be prevented. However the hydrates may alter the rheologi of the mixture and consequently influence the capacity of the transport system.

A third approach, which is described in U.S. Pat. No. 6,774,276 B1, comprises a so called cold flow method, where a hydrate slurry is formed, an a part of this is taken out, returned and is injected into the flow at a point prior to the point where the hydrate formation occurs. The particles in the slurry function as condensation seeds and pick up water as they grow bigger; this method also implies a rapid cooling of the flow just after the well head.

Other prior solutions to prevent or control hydrate formation are known i.a. from the following publications: U.S. Pat. No. 6,417,417, EP 1 561 069 and WO 2005/000746.

WO 2007/095399 teaches a method for creating a non-plug forming hydrate slurry. This publication describes a hydrate controlling transport solution where a part of the main flow is diverted to a “cold flow” reactor to produce endogenic “dry” small hydrate particles when they cross static mixers, before they are reinjected into the main flow. The advantage of this solution is that by the production no energized equipment (such as pumps) is necessary and the amount of dry hydrate seeds cannot assures, since the dimensioning is fixed, while the characteristics of the transported hydrocarbons varies, and the hydrate seeds must be present in the hydrocarbon flow, i.e. are endogenic. Consequently the quality of the hydrate control will vary. This solution require that the temperature is within a certain range for the hydrate seeds to survive, and must not reach the hydrate condition to far before the injection point, since this can result in plug formation. The temperature profile along the pipeline can be altered as a function of the production amount, the sea temperature (seasons) and of the production changes as a consequence of the age of the field, e.g. with increasing water content.

From WO 2002/1027 it is known a hydrate control transport system for a fluidised bed heat exchanger which acts as an endogenic generator for hydrate particles, for thereby stabilising the hydrocarbon flow as a gas hydrate slurry before its pumped away in a pipeline. This solution implies “energized” equipment and a comparatively complex fluidised bed subsea heat exchanger, which thereby reduces the toughness of the solution. Additionally, since the seeds are endogenic (and not added under control) and the handling of operation of such installations can be highly complex, especially when the inlet flow has variable characteristics, the quality of the hydrate control will be variable.

From WO 2008/056250 it is known a hydrate control transport solution where a cooling circuit functions as an endogenic generator for macerating hydrate particles after reinjection into the main flow, for thereby stabilise the hydrocarbon flow as a gas hydrate slurry before it is pumped away in the pipeline. This solution implies use of “energised equipment”, such as a macerator. Additionally, since the seeds are endogenic (and are not added under control), and the handling of the operation of such solutions can be relatively complex, and especially when the incoming flow has variable characteristics, the hydrate control can be variable. This implies mechanical equipment which is not appropriate subsea, especially at large depths. Wear due to abrasion and crushing will require maintenance. It also requires that the temperature reaches a certain level at a certain position, which is not always a simple matter, as discussed. The equipment can get in the way for pigging operations.

A characteristic phenomenon as mentioned above is that the hydrates are not formed instantly when the fluids enter the area for hydrate formation. The hydrate formation rate will depend on the degree of sub cooling (“distance from the hydrate formation limit”) and the time since passing below the hydrate formation temperature. This is designated as the hydrate kinetic problem. A decisive factor to determine this time delay is the presence of seed particles.

The idea on which the present invention is based, is to add seed particles which are much more effective to promote hydrate formation than the pipe wall, and consequently take up water into smaller particles before it get the possibility to form hydrates on the pipe wall.

It is assumed that the most effective seed particles probably are hydrate surfaces. In U.S. Pat. No. 6,774,276 B1 mentioned earlier, it is proposed to use this by recycling some of the hydrate slurry which has been captured downstream of the hydrate formation point. Additionally this slurry is cold after being cooled in an annulated recirculation branch. In this way, the main flow will flow quicker through the hydrate region and the hydrate formation is promoted at a distance from the pipe wall. This requires a quite expensive subsea installation to separate a appropriate slurry quantity, transport it back to the point of injection and inject. This installation may complicate the necessary operations, i.e. pigging. Further it will be difficult to obtain the required balance between cooling before the injection point and the quantity of re-injected hydrate slurry. It will be desirable to bring the production flow close to the hydrate formation point in such a way that injected, cold hydrate brings the mixture below the hydrate formation point. Conjunction of unfortunate circumstances can result in to much cooling of the mixture and form hydrate blocking before the injection point. Unfortunate conjunctions can be low production rate, and low sea temperature, strong ocean currents. The temperature profile through the pipeline will not be constant, but will vary with production rate and external cooling of the pipeline. At high production rates, the temperature will stay high a longer distance from the well head. The production rate can be altered due to market demand, functional problems in wells or receiving installations.

The purpose of present invention is to modify this procedure by injecting condensations seeds from outside. The condensation core can be solid particles as silver iodide or other solid compounds with low solubility, with an ice-like crystal structure, or mineral particles such as light metal oxides, e.g. titanium oxide or aluminium oxide, or hydrate particles. Tests with hydrate formation in a small reactor with stirring show that both titanium oxide and silver iodide reduce the time from the point where the conditions for hydrate formations has been reached, until the actual hydrate formation has been observed, with a factor of approx. 60. Hydrate formation is then observed in that gas is absorbed into the hydrate structure, in such a way that a considerable pressure reduction is observed. The hydrate particles shall possibly be formed with a gas that can stabilise the hydrates at higher temperatures and lower pressure than the natural gas in the transport system. A series of gases and volatile liquids form hydrates at substantially lower pressure and higher temperature than natural gas. Examples can be tetrahydrofuran, sulphur hexafluoride, and several halogenated hydrocarbons. Thus a possibility can be to use tetrahydrofuran which forms hydrates very easily at low pressure and relatively high temperature.

The mineral particles should be low solubility, non-reactive particles and hydrophilic with OH- or O-terminated groups on the surface. The O-group will form OH-groups by special preparation or in contact with water. In this way, they can be injected a certain distance from the point where the main flow reaches the hydrate formation region and one can be pretty sure that they have survived and are active for water uptake. This gives a greater liberty regarding the design of such a system, since it is not necessary to map the point where the mixture enters the hydrate formation region. The seeds can be injected well before the point where the system goes into the region where the natural gas hydrates are thermodynamically stable. Due to the survival ability of the seeds, they will be able to absorb water when the thermodynamic conditions allow hydrate formation.

Cooling will increase the viscosity of the oil to a smaller or larger degree, depending on the actual type of oil. This will increase the flow resistance, much of which depends upon the Reynold's number for the flow. Another effect that can increase the flow resistance by cooling is the actual formation of particles; these will alter the rheologi of the slurry and increase the viscosity more that corresponding volume fraction of droplets will do. It is therefore a desire to postpone the cooling as far as possible, and preferably as in the present invention, not provoke it.

When the hydrates are formed without the aid of seeds, it is assumed that the formation rate will be limited by the availability of (stabilising) gas. This indicates that water droplets start to form hydrates in an outer shell. This means that the availability of gas to the inner part of the droplet is restrictive, and hydrate particles with a wet centre can be achieved.

Such particles can break up by impingement with the pipe wall or other particles. The water and the gas will be available exactly at the point in time where a particle is close to another surface, and the subsequent hydrate formation will probably be able to form a bridge between the particle and the surface.

On the other hand, if the particles are growing from a seed, they will grow from the inside of a droplet and the hydrate will not obstruct the gas to reach the remaining water and the hydrate formation can tie up all the water. It can be a vulnerable period until all the water has been transformed and where bonds to other surfaces can be formed. If the seeds are sufficiently effective, such cases would be rare.

Nerheim et al. (Laser light scattering studies of natural gas hydrates, SPE Annual Technical Conference and Exhibition, Volume Sigma, Issue pt2, 1994, pp-303-307) have found that seed particles must have a certain minimum size to be effective. This size seems to be in the region 5-30 nm.

As mentioned, seed particles can be pure gas hydrates or particles such as silver iodide with ice-like crystal structure and which function as seed formers both for ice (rain making) and hydrate formation (Wilson, P. W, Lester, D. and Haymet, heterogeneous nucleation of clathrates from super cooled tetrahydrofuran/THF/water mixtures, and the effect of an added catalyst, Chemical Engineering Science, Vol. 60, Issue 11, June 2005, pp. 2937-2941). In the case of heavy particles such as silver iodide, it can be beneficial to affix them to the surface of larger, lighter particles (plastic) in order to ensure a easier mixture of them with the oil and water, and not sink to the bottom. IN the case of gas hydrates, there is a possibility to control the particle size by crushing or by extensive mixing/turbulence during the formation of the hydrate seeds, in order to obtain a particle size typical in the region of 0.1 to 2 micrometers.

In the case of silver iodide that shall be affixed to lighter plastic particles, it is conceivable to imagine porous particles saturated with silver nitrate which are transferred to a ammonium iodide solution. Silver ions and iodide ions will then react with each other and form insoluble silver iodide crystals on the surface of the plastic particles.

The silver iodide particles have the advantage that the can be captured and recuperated if desired. Those who avoid such a recuperation will be biological inert, i.e. will not be taken up into the food chain, since they are not soluble in water or fat.

There is probably an optimal size for the hydrate particles to be transported, small enough to be carried away in the flow, but sufficiently small so that the specific surface will not be too large and cause an unwanted increase of the rheological parameters such as yield stress or consistence factor/viscosity. The particle size will be a function of the amount of available water in relation to the number of seed particles. Consequently a possibility will be to produce the sufficient number of seed particles giving the optimum size of the hydrate particles.

If one could produce hydrate seed particles which are 1/10 of the desired size of the final transportable particles, the water used for producing seed will carry 10³ times the amount of produced water. Consequently the added water will not represent any additional load for the transport- or the separation system.

An possible method for producing hydrate seeds which are stable at relatively high temperatures, is to use tetrahydrofuran as the hydrate stabilising molecule. There are also other usable gases. The inventor of the present invention has used gases of the Freon type to produce hydrates at low pressures, and these are possible candidates, but however they are probably not appropriate from an environmental point of view.

The method for producing hydrate seed particles must fulfil two requirements:
They must block themselves during the hydrate formation process; and
Sufficiently small seeds must be produced in order to produce a sufficient number of seed with little amount of water.

The ratio between the number of seeds and the amount of water to be taken up must be correct. Too few seeds and the particles will be large and have a greater tendency for sinking and layer formation, which require a higher flow rate of the mixture to purge them out. With too many seeds, the water will be distributed between many, small particles; the result is a large specific surface, and the small particles will have a larger tendency to stick together and the slurry will have a more unfavourable rheology than with appropriate size.

We will assume that seed particles seed particles are formed of water, oil and the gas in question. Further that these liquids are cooled to the temperature of the water surrounding the transport pipeline on ocean floor, typically 4 to 6° C. Consequently these fluids will lie within the region of hydrate formation at the typical injection pressure.

Based on the above considerations, the purpose of present invention is to provide a more effective and environmental friendly method for promoting the formation of gas hydrates.

This purpose is obtained by a method for the production of seed particles which is characterised by the features of the enclosed patent claims. The invention is also directed to use of silver iodide and hydrate particles formed by use of a more effective hydrate forming gas, respectively, according to the independent use claims.

Use of metal oxides is also conceivable, especially titanium dioxide or aluminium oxide. These are supposed to have a small negative environmental effect. They can further advantageously be recuperated, e.g. by filtering, but due to the low price, it is also conceivable to be omitted.

In the following the invention will be explained more in detail by different embodiments and with reference to the appended drawing. The drawing shows schematically a subsea pipeline carrying a hydro carbon fluid/water-mixture.

### EXAMPLE 1

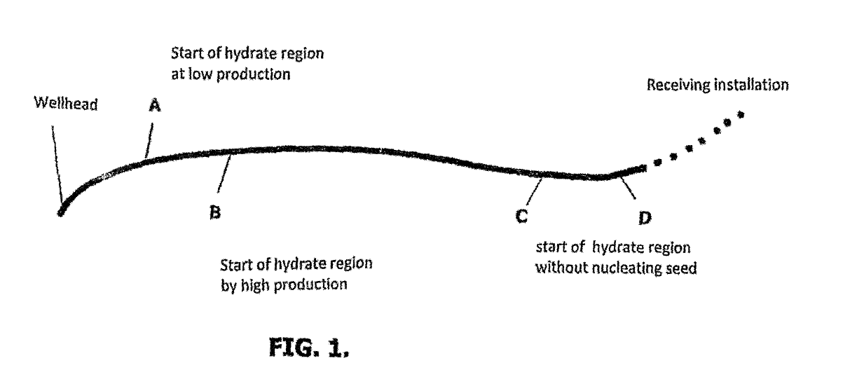

The method is considered used on a subsea transport pipeline for gas condensate leading from a well head manifold to a receiving installation onshore, as schematically shown in FIG. 1. At low production and low sea temperature the condition for hydrate formation is reached at point A, while at high production and high sea temperature this condition is reached at point B. The seed former is silver iodide on plastic spheres, in such a way that they have a density close to paraffin (kerosene). In order not to stress the water separation plant at the receiving installation, the plastic spheres are mixed with paraffin at a concentration which does not increase the viscosity of the mixture too much. The paraffin/seed former-sphere mixture is pumped through a service pipeline to the well manifold and is injected into the production pipeline just after the well manifold. Then the injection point is far ahead of point A where the seed formers at worst must be injected and mixed into the production flow. The diameter of the service pipeline is adapted to the amount of seed former slurry in order to obtain an appropriate turbulent flow that can transport the plastic spheres. The turbulence does not have to be severe, since the spheres have an approximately neutral buoyancy.

Corrosion inhibitors that stabilise hydrocarbons present on the surface have typically been added to the flow of oil, and consequently the hydrophobic behaviour of the surface increases. Consequently the probability of the hydrate particles to attach to the pipe wall will be lesser than in a system without surface active compounds.

Seed formers follow the flow which is quite turbulent due to the low viscosity of the condensate and produced water dispersed in this.

The seed particles will in a large extent be trapped in the interface between oil and water. At point B the formation of hydrates around the seed particles starts. The latent heat liberated by the hydrate formation, will decrease the cooling of the production flow.

The hydrate particles and the remaining water are separated from oil and gas. After pressure relief the hydrates dissociates, the released gas is recycled to the process. If desired, the seed particles can be recuperated by filtering out the particles or by capture with a magnet in that the plastic particles to which they are attached, contain a super paramagnetic particle (this is particles containing a small amount of chaotic arranged magnetic crystallites, i.e. having no outer magnetic momentum and do not attach themselves to the steel wall of the tube but in an external magnetic field the magnetic momentum are directed according to the field, and the particle is attracted by the magnet. This is a well known method for separation of so-called Ugelstad-spheres within the field of biochemistry). The recuperation can be done after pressure relief both for the water phase and the condensate phase.

By altering the surface characteristics of the steel, the risk of particles attaching themselves to the pipe wall can be reduced. By use of ordinary CO₂— corrosion inhibitors, it is i.a. possible to stabilise an oil film which is present on the steel surface. Consequently this will prevent that hydrophilic particles such as hydrates, or water emulsions will adhere to the surface.

### EXAMPLE 2

### Use of TiO₂ as Nucleating Particles

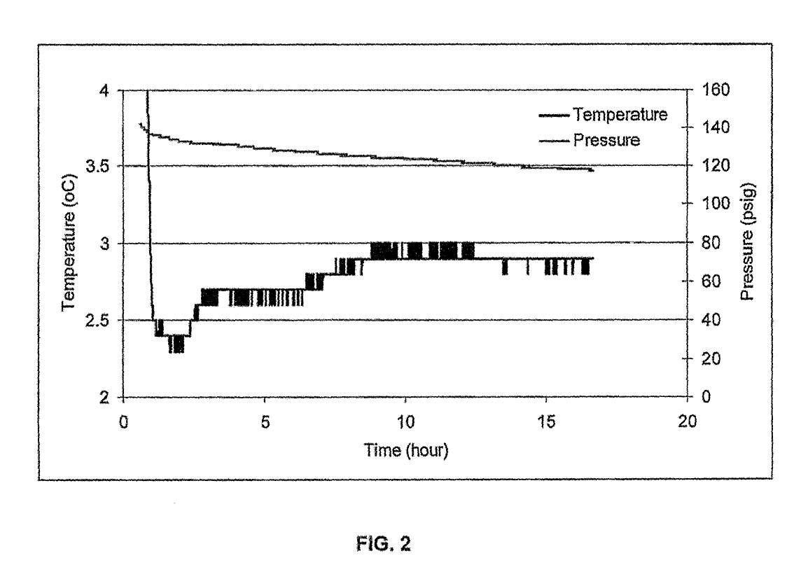

FIG. 2 shows pressure and temperature progress in the reactor with TiO₂ as nucleating particles. It is evident that initially the temperature control system cools the reactor down to the set point 1.5° C., the pressure decreases since the gas is being cooled. After about 2 hours the experiment starts and the reaction heat makes then temperature to rise. Due to the absorption of gas into the hydrate structure, the pressure decreases even though the temperature increases. The hydrate formation take place for a long time until all the water is bound; the reason for this is probably that we have too few and too large TiO₂ particles. Without artificial nucleating particles, it last 4-5 days until we can observe hydrate formation, in contrast to 2 hours here (in reality it is rather half an hour from reaching hydrate conditions, about 4° C., until the hydrate formation starts). The absolute period of time is not representative for what is happening in pipe flow, better mixing will result in shorter time from hydrate conditions until hydrate actual is formed, but this relative relationship should show how much more effective the oxide particles are to promote hydrate formation than the steel wall is.

We claim:

1. Method for adding nucleation seeds to promote formation of transportable hydrate particles in a flow comprising hydrocarbon fluid and water, characterized in adding nucleation seeds at a point in the flow before the hydrate forming region, whereby the nucleation seeds are available for promoting hydrate formation when said region is reached.

2. Method according to claim 1, characterized in that the flow is a fluid flow in a subsea transport pipeline.

3. Method according to claim 1, characterized in that the nucleation seeds comprises silver iodide (AgI) particles.

4. Method according to claim 3, characterized in that the silver iodide particles are larger than 30 nm.

5. Method according to claim 1, characterized in that the nucleation seeds are fixed to a medium with a lower density that the surrounding fluid flow.

6. Method according to claim 5, characterized in that the medium is plastic spheres with a diameter in the area from 1-10 μm, possibly containing super paramagnetic particles.

7. Method according to claim 1, characterized in that the nucleation seeds comprises particles produced of a gas that gives a hydrate with a melting temperature that is higher than the temperature of the surrounding fluid flow.

8. Method according to claim 1, characterized in that the nucleation seeds are tetrahydrofuran.

9. Method according to claim 1, characterized in that the nucleation seeds are mineral particles.

10. Method according to claim 9, characterized in that mineral particles have low solubility, are particles with low reactivity and hydrophilic with OH- or O-terminated surface groups.

11. Method according to claim 9, characterized in that mineral particles are light metal particles, for example titanium dioxide or aluminium oxide.

12. Use of silver iodide as nucleating seeds for promoting hydrate formation in a fluid flow containing hydrocarbon fluids and water.

13. Use of tetrahydrofuran-hydrate in particle form as nucleating seeds to promote hydrate formation in a fluid flow containing hydrocarbon fluids and water.

14. Use of mineral particles with low solubility, said metal particles are non-reactive particles and hydrophillic with OH- or O-terminated surface groups such as light metal oxides, for example titanium dioxide or aluminum oxide, to promote hydrate formation in a fluid flow containing hydrocarbon fluids and water.

15. Method according to claim 10, characterized in that mineral particles are light metal particles, for example titanium dioxide or aluminium oxide.
